(19) United States
(12) Patent Application Publication
(10) Pub. No.: US 2006/0246143 A1
(43) Pub. Date: Nov. 2, 2006

(54) Targeted therapy via targeted delivery of energy susceptible nanoscale magnetic particles

(76) Inventor: Hilmi Ege, Rochester, MN (US)

Correspondence Address: Hilmi Ege, M.D., 621 1st Street S.W., Apt. No: 306, Rochester, MN, 55902, US

(21) Appl. No.: 11/410,607

(22) Filed: Apr. 25, 2006

Related U.S. Application Data
(60) Provisional application No. 60/675,970, filed on Apr. 28, 2005.

(51) Int. Cl. A61K 48/00; A61K 9/14

(52) U.S. Cl. 424/489; 514/44; 977/916

(57) ABSTRACT

The present invention relates generally to targeted therapy with RNA interference, more specifically, to energy susceptible nanoscale material compositions, devices for use with magnetic material compositions, and methods related thereto for targeted therapy via targeted delivery of energy susceptible nanoscale magnetic particles carrying short interfering RNA constructs.

### CROSS REFERENCE TO RELATED APPLICATION

This is a non-provisional application claiming the benefit of and priority to a provisional patent application with Ser. No. 60/675,970 filed on Apr. 28, 2005.

### BACKGROUND OF THE INVENTION

The time between the onset of disease in a patient and the conclusion of a successful course of therapy is often unacceptably long and expensive. Many diseases remain asymptomatic and evade detection while progressing to advanced and often terminal stages. In addition, this period may be marked by significant psychological and physical trauma for the patient due to the unpleasant side effects and complications of even correctly prescribed treatments. Even those diseases that are detected early may be most effectively treated only by therapies that disrupt the normal functions of healthy tissue or have other unwanted side effects.

One such disease is cancer. Despite considerable research effort and some success, cancer is still the second leading cause of death in the United States, claiming more than 500,000 lives each year according to American Cancer Society estimates. Traditional treatments are invasive and/or are attended by harmful side effects (e.g., toxicity to healthy cells), often making for a traumatic course of therapy with only modest success. Early detection, a result of better diagnostic practices and technology, has improved the prognosis for many patients. However, the suffering that many patients must endure makes for a more stressful course of therapy and may complicate patient compliance with prescribed therapies. Further, some cancers defy currently available treatment options, despite improvements in disease detection. Of the many forms of cancer that still pose a medical challenge, prostate, breast, lung, colon and liver claim the vast majority of lives each year. Colorectal cancer, ovarian cancer, gastric cancer, leukemia, lymphoma, melanoma, and their metastases may also be life-threatening.

Conventional treatments for breast cancer, for example, typically include surgery followed by radiation and/or chemotherapy. These techniques are not always effective, and even if effective, they suffer from certain deficiencies. Surgical procedures range from removal of only the tumor (lumpectomy) to complete removal of the breast. In early stage cancer, complete removal of the breast provides the best assurance against recurrence, but is disfiguring and requires the patient to make a very difficult choice. Lumpectomy is less disfiguring, but is associated with a greater risk of cancer recurrence. Radiation therapy and chemotherapy are arduous and are not completely effective against recurrence.

Treatment of pathogen-based diseases is also not without complications. Patients presenting symptoms of systemic infection are often mistakenly treated with broad-spectrum antibiotics as a first step. This course of action is completely ineffective when the invading organism is viral. Even if a bacterium (e.g., E. coli) is the culprit, the antibiotic therapy eliminates not only the offending bacteria, but also benign intestinal flora in the gut that are necessary for proper digestion of food. Hence, patients treated in this manner often experience gastrointestinal distress until the benign bacteria can repopulate. In other instances, antibiotic-resistant bacteria may not respond to antibiotic treatment. Therapies for viral diseases often target only the invading viruses themselves. However, the cells that the viruses have invaded and “hijacked” for use in making additional copies of the virus remain viable. Hence, progression of the disease is delayed, rather than halted.

For these reasons, it is desirable to provide improved and alternative techniques for treating disease. Such techniques should be less invasive and traumatic to the patient than the present techniques, and should only be effective locally at targeted sites, such as diseased tissue, pathogens, or other undesirable matter in the body. Preferably, the techniques should be capable of being performed in a single or very few treatment sessions (minimizing the need for patient compliance), with minimal toxicity to the patient. In addition, the undesirable matter should be targeted by the treatment without requiring significant operator skill and input.

Genetic expression profiling of tumors for more targeted therapies are rapidly expanding. Better prognostication of cancers and determination of active pathways sustaining the malignant growth is possible. Another emerging field is cancer proteomics which helps the physician determine the proteins which are the end products of these active pathways. It is possible to postulate which pathways present in the cancer cell might be active and contributing to the malignant growth by looking at the genetic expression profiles and proteomic analysis of the cancer cells.

Our ability to construct nucleotide sequences at a specifically desired length, order and composition has also remarkably improved. The cost of this procedure has also come down quite significantly in the last few years. It is now possible to manufacture a desired specific nucleotide sequence for a clinical use purpose at a very cost effective way. With the rapid development of technology in the field, it is discovered that various short interfering ribonucleic acid (siRNA) and micro interfering (mRNA) oligonucleotide constructs can downregulate or upregulate their targeted pathways. This is thought to be a part of genetic regulation mechanism. There are ongoing clinical trials in humans to benefit from this regulatory mechanism by downregulating the active pathways contributing to the malignant growth.

Malignant growth is usually sustained by the contribution of multiple active pathways inside a cancerous cell. Recent cancer therapies are targeting these pathways and named as such targeted therapies. Such a successful targeted therapy can be against the fusion protein product. Imatinib is an active targeted therapy agains the fusion protein endproduct of a translocation resulting in malignancy. Clinical data is pointing towards mulple active cellular pathways and compensatory mechanisms working inside both normal and cancerous cells. A single targeted therapy against a single pathway may not give the final durable endresult but a multitargeted approach, attacking multiple active pathways inside a cancer cell may bring down the intracellular network keeping the cancer cell alive. Multitargeted therapies are currently in development.

One disadvantage of such multitargeted therapies will be the expense of them. They are costly to develop and manufacture. They are relatively limited in the forms of monoclonal antibodies and small molecules. However, it is possible to take another method of approach to downregulating active cellular pathways of a cancer cell by using nature's own regulatory mechanism, short interfering ribonucleic acid (siRNA) and micro interfering (mRNA) oligonucleotides. One shortfall of this approach has been the difficulty of delivering the oligonucleotides to the target tissue before they are destroyed and the limited quantity that reaches the target tissue after a systemic delivery of them.

This can be overcome by using energy susceptible nanoparticles with magnetic properties which may be directed to specific body site or organ with the use of a magnetic field. Furthermore delivery of the short interfering ribonucleic acid (siRNA) and micro interfering (mRNA) oligonucleotides can be achieved with energy transfer to the energy susceptible nanoscale particles once they are at the target area.

One other advantage of this system will be combined effect of oligonucleotide sequences on the cellular pathways and energy transfer into the cell causing hyperthermia and mechanical disruption of intracellular machinery. The goal of the system will be causing apoptosis of the cancer cell. This medical device and system can also be used against various pathogens such as bacteria, viruses, parasites and fungi. Oligonucleotide sequences specifically designed to disrupt their viability networks can be constructed and delivered to their location. Biological information is stored and used as nucleotide sequences. This medical device and system will bring the level of therapy to an information war or misinformation war to disrupt the viability pathways and networks of pathogens.

Temperatures in a range from about 40.degree. C. to about 46.degree. C. (hyperthermia) can cause irreversible damage to disease cells. However, healthy cells are capable of surviving exposure to temperatures up to around 46.5.degree. C. Diseased tissue may be treated by elevating the temperature of its individual cells to a lethal level (cellular thermotherapy). Pathogens implicated in disease and other undesirable matter in the body can be also be destroyed via exposure to locally-high temperatures.

Hyperthermia may hold promise as a treatment for cancer because it induces instantaneous necrosis (typically called “thermo-ablation”) and/or a heat-shock response in cells (classical hyperthermia), leading to cell death via a series of biochemical changes within the cell. State-of-the-art systems that employ radio-frequency (RF) hyperthermia, such as annular phased array systems (APAS), attempt to tune E-field energy for regional heating of deep-seated tumors. Such techniques are limited by the heterogeneities of tissue electrical conductivity and that of highly perfused tissue. This leads to the as-yet-unsolved problems of “hot spot” phenomena in untargeted tissue with concomitant underdosage in the desired areas. These factors make selective heating of specific regions with such E-field dominant systems very difficult.

Another strategy that utilizes RF hyperthermia requires surgical implantation of microwave or RF based antennae or self-regulating thermal seeds. In addition to its invasiveness, this approach provides few (if any) options for treatment of metastases because it requires knowledge of the precise location of the primary tumor. The seed implantation strategy is thus incapable of targeting undetected individual cancer cells or cell clusters not immediately adjacent to the primary tumor site. Clinical success of this strategy is hampered by problems with the targeted generation of heat at the desired tumor tissues.

### BRIEF SUMMARY OF THE INVENTION

The present invention relates generally to targeted therapy, more specifically, to energy susceptible nanoscale material compositions, devices for use with magnetic material compositions, and methods related thereto for targeted therapy via targeted delivery of energy susceptible nanoscale magnetic particles.

Hyperthermia for treatment of disease using magnetic fluids exposed to RF fields has been recognized for several decades. However, a major problem with magnetic fluid hyperthermia has been the inability to selectively deliver a lethal dose of particles to the cells or pathogens of interest.

An emerging field is gene expression regulation with short interfering ribonucleic acid (siRNA) and micro interfering (mRNA) oligonucleotide constructs. It is possible to downregulate or upregulate desired genes with short interfering ribonucleic acid (siRNA) and micro interfering (mRNA) oligonucleotide constructs. Delivery of these constructs to the target tissue has been a challenge.

In view of the above, there is a need for a method for treating diseased tissue, pathogens, or other undesirable matter, that incorporates selective delivery of energy susceptible nanoscale magnetic compositions with short interfering ribonucleic acid (siRNA) and micro interfering (mRNA) oligonucleotide constructs under magnetic fields to a predetermined target within a patient's body. It is also desirable to have treatment methods that are safe and effective, short in duration, with minimal side effects and require minimal invasion.

It is, therefore, an object of the present invention to provide a medical device with a treatment method that involves the administration of energy susceptible nanoscale size particles with magnetic material compositions, which contain single-domain magnetic particles attached to short interfering ribonucleic acid (siRNA) or micro interfering (mRNA) oligonucleotide constructs to a patient and the application of energy by an energy source such as an alternating magnetic field to inductively heat the energy susceptible magnetic material composition. Energy delivery will help release and unleash short interfering ribonucleic acid (siRNA) or micro interfering (mRNA) oligonucleotide constructs to the predetermined target area. Heating and motion produced by the energy delivered to the nanoparticle will release the short interfering ribonucleic acid (siRNA) or micro interfering (mRNA) oligonucleotide constructs to the predetermined target area.

It is another object of the present invention to provide such a treatment method that includes the detection of at least one location of accumulation of the magnetic material composition within the patient's body prior to the application of an alternating magnetic field.

It is another object of the present invention to provide such a treatment method that involves the application of the energy source such as alternating magnetic field when the energy susceptible nanoscale size magnetic material compositions, which contain single-domain magnetic particles attached to short interfering ribonucleic acid (siRNA) or micro interfering (mRNA) oligonucleotide constructs are outside the patient's body.

It is yet another object of the present invention to provide a method for administration of the energy susceptible nanoscale size magnetic material compositions, which contain single-domain magnetic particles attached to short interfering ribonucleic acid (siRNA) or micro interfering (mRNA) oligonucleotide constructs, which may be intraperitoneal injection, intravascular injection, intramuscular injection, subcutaneous injection, topical, inhalation, ingestion, rectal insertion, wash, lavage, rinse, or extracorporeal administration into patient's bodily materials.

It is a further object of the present invention to provide methods for the treatment of tissue in a safe and effective manner, with minimal invasion, and short treatment periods.

The present invention pertains to methods for treating disease material in a patient. In one embodiment, a treatment method is disclosed that involves the administration of energy susceptible nanoscale size magnetic material compositions, which contain single-domain magnetic particles attached to short interfering ribonucleic acid (siRNA) or micro interfering (mRNA) oligonucleotide constructs, to a patient and the application of an alternating magnetic field to inductively heat the magnetic material composition.

In another embodiment, a treatment method is disclosed that involves the administration of energy susceptible nanoscale size magnetic material compositions, which contain single-domain magnetic particles not attached to short interfering ribonucleic acid (siRNA) or micro interfering (mRNA) oligonucleotide constructs, detecting at least one location of accumulation of the magnetic composition within the patient's body, and the application of an alternating magnetic field to create motion of the magnetic nanoparticle leading to the release of short interfering ribonucleic acid (siRNA) or micro interfering (mRNA) oligonucleotide constructs. In this embodiment, magnetic nanoparticles and short interfering ribonucleic acid (siRNA) or micro interfering (mRNA) oligonucleotide constructs are inside a biodegradable sphere. They do not need to be attached. Motion and heat created by the energy transfer to the magnetic nanoparticles by electromagnetic waves release the contents of the biocompatible sphere as the motion and heating of the energy susceptible nanoparticles disrupt the biocompatible sphere.

In another embodiment, a treatment method is disclosed that involves the administration of the energy susceptible nanoscale size magnetic material compositions, which contain single-domain magnetic particles attached to short interfering ribonucleic acid (siRNA) oligonucleotide constructs to a patient, and application of an alternating magnetic field to induce a desired pathological effect by inductively heating the thermotherapeutic magnetic nanoparticle to release the attached short interfering ribonucleic acid (siRNA) oligonucleotide constructs which will cause a necrosis, an apoptosis, or a pathogen deactivation.

In another embodiment, a treatment method is disclosed that involves the administration of the energy susceptible nanoscale size magnetic material compositions, which contain single-domain magnetic particles attached to short interfering ribonucleic acid (siRNA) oligonucleotide constructs, which may be intraperitoneal injection, intravascular injection, intramuscular injection, subcutaneous injection, topical, inhalation, ingestion, rectal insertion, wash, lavage or rinse perisurgically, or extracorporeal administration into patient's bodily materials.

Any of the disclosed embodiments may include treatment methods including monitoring of at least one physical characteristic of a portion of a patient.

Any of the disclosed embodiments may include treatment methods where the predetermined target is associated with diseases, such as cancer, diseases of the immune system, and pathogen-borne diseases, and undesirable targets, such as toxins, reactions to organ transplants, hormone-related diseases, and non-cancerous diseased cells or tissue.

The above summary of the present invention is not intended to describe each illustrated embodiment or every implementation of the present invention. The figures and the detailed description which follow particularly exemplify these embodiments.

### BRIEF DESCRIPTION OF THE SEVERAL VIEWS OF THE DRAWING

The invention may be more completely understood in consideration of the following detailed description of various embodiments of the invention in connection with the accompanying drawings, in which:

FIG. 1 schematically illustrates a medical device and treatment system according to an embodiment of the present invention;

FIG. 2 schematically illustrates a targeted treatment according to an embodiment of the present invention;

FIG. 3 schematically illustrates a circuit for producing an alternating magnetic field according to an embodiment of the present invention;

FIG. 4a graphically illustrates a therapeutic sinusoidal current waveform according to an embodiment of the present invention;

FIG. 4b graphically illustrates a therapeutic triangular current waveform according to an embodiment of the present invention;

FIG. 5a graphically illustrates a therapeutic sinusoidal waveform modulation according to an embodiment of the present invention;

FIG. 5b graphically illustrates a therapeutic pulsed waveform modulation according to an embodiment of the present invention;

FIG. 6 schematically illustrates a handheld medical device and therapy system configuration according to an embodiment of the present invention;

FIG. 7 schematically illustrates the procedural steps according to an embodiment of the present invention;

FIG. 8 schematically illustrates nanoscale paricles outside and inside of a disease cell according to an embodiment of the present invention;

FIG. 9 schematically illustrates a composition of nanosirna according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE INVENTION

The present invention pertains to individualized treatment by RNA interference with use of short interfering RNAs (siRNA) incorporated into nanoparticles with magnetic material compositions, medical devices for treating diseased, disease-causing, or undesirable tissue or material, for use with magnetic nanoparticle material compositions, delivery of siRNAs to predetermined target areas and methods for treating the tissue or material utilizing such devices and nanoscale magnetic energy susceptor material compositions. Diseased, disease-causing, or undesirable material in the body are referred to herein as “disease material”. It is a main object of this invention to treat cancer on an individualized basis with the use of gene expression profile of the cancer. The therapeutic methods disclosed herein include the targeted delivery of nanometer sized magnetic particles carrying and delivering siRNAs to the target material. The term “nanosirna”, as used herein, refers to the composition including a nanoscale magnetic energy susceptor core particle, a biocompatible coating material, and short interfering RNAs (siRNA). Short interfering RNAs are selected based on the gene expression profiling of the predetermined target. Predetermined target can be cancer. Proteomic analysis of the predetermined target can also help in choosing the siRNA constructs. Multiple siRNA constructs of different or same type can be incorporated into these nanoscale size magnetic particles. SiRNA constructs may be chemically modified. The methods for treating disease material disclosed herein include administering to a patient the nanosirnas suspended in an appropriate medium, and applying, via a device capable of interacting with the nanosirnas, a magnetic field to an area of the patient containing the predetermined target, which can be cancer, to increase their concentration at the predetermined target area. Then, applying an alternating magnetic field to the nanosirnas to create their motion and heat them to cause release of siRNAs that they are carrying which kill or render ineffective the disease material. A magnetic field is necessary to concentrate nanosirnas at the site of the predetermined target. This magnetic field may be static or alternative magnetic field. However, after this step, energy transfer to the nanosirnas may be achieved by infrared light or any other type of electromagnetic wave. One example can be nanoshells which absorb energy from infrared light. This energy transfer creates heat and motion of the nanosirnas, leading to the release of siRNAs that they are carrying to the environment. Mechanical motion of the nanosirnas also cause mechanical disruption in their surrounding tissue and the heat generated by the energy transfer via electromagnetic waves from the energy source further causes destruction of the predetermined target.

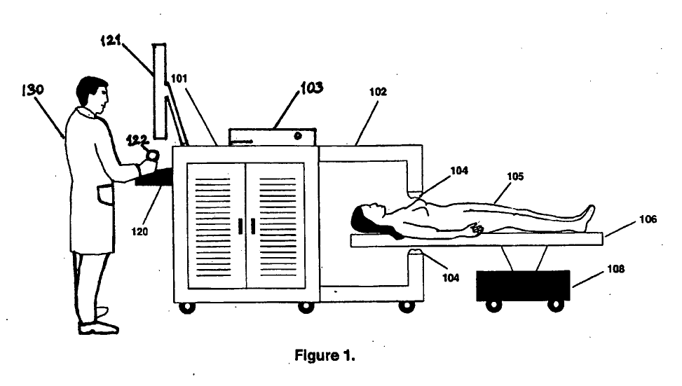

One embodiment of the invention, as illustrated in FIG. 1, includes an electromagnetic wave generator in the form of an alternating magnetic field (AMF) generator located within a cabinet 101 designed to produce an alternating magnetic field (AMF) that may be guided to a specific location within a patient 105 by a magnetic circuit 102. The therapeutic methods of the present invention may be performed following a determination of the presence of disease material in one or more areas of the patient. For example, the disease material may be any one or combination of cancers and cancerous tissue, a pathogenic infection (viral, bacterial or multicellular parasitic), toxin, or any pathogen-like material (prion). The manner of making the diagnosis does not form part of the invention and may be performed using any standard method. However, the present invention, or aspects thereof, may be amenable to a diagnostic function alone or in conjunction with another method or apparatus. Such a diagnostic function would be performed by using a suitable technology or technique to interrogate the magnetic properties of the nanosirnas, and thus evaluate their concentration and location within the patient. Both the location and concentration of nanosirnas may be determined using an existing technique such as magnetic resonance imaging, or another diagnostic technique can be established and performed using a suitable magnetometer, such as a Superconducting Quantum Interference Device (SQUID). Information obtained from this interrogation may be used to define the parameters of treatment, i.e. the location, duration, and intensity, of the alternating magnetic field. The patient lies upon an X-Y horizontal and vertical axis positioning bed 106. The bed 106 is both horizontally and vertically positionable via a bed controller 108. The AMF generator produces an AMF in the magnetic circuit 102 that exits the magnetic circuit at one pole face 104, passing through the air gap and the desired treatment area of the patient, and reenters the circuit through the opposing pole face 104, thus completing the circuit. An operator or medical technician 130 is able to both control and monitor the AMF characteristics and bed positioning via the control panel 120. The operator 130 may use a computer 103 and visualize the position of pole face 104 relative to the predetermined target disease material by using a computer monitor 121. The operator may position the magnetic circuit 102 to the desired coordinates by using a joystick controller 122.

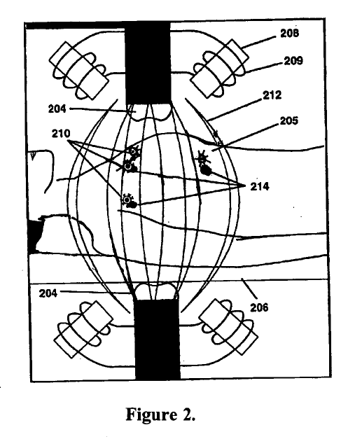

FIG. 2 illustrates a treatment of a patient with a device for treating disease material according to an embodiment of the present invention. The area of the patient to be treated 205 is localized in the region between the magnetic poles 204 via the positionable bed 206. This region may be any location of the patient including the chest, abdomen, head, neck, back, legs, arms, any location of the skin. An AMF may be applied to the treatment area 205 of the patient, as illustrated by the magnetic lines of flux 212. The magnetic field, manifested by the magnetic lines of flux 212 interacts with both healthy and disease material in the localized area. Nanosirnas 210, containing at least one appropriate siRNA selected based upon the gene expression profile of the particular type of disease material, are concentrated to the diseased area by applying a magnetic field to the diseased area 214. In the illustrated case, the predetermined target is metastatic lung cancer and the nanosirnas 210 carry siRNA constructs selected based upon the gene expression profile of this particular metastatic lung cancer. The nanosirnas 210 become excited by the interacting with applied AMF which creates motion of them and they are also inductively heated to a temperature sufficient enough to lead to the release of siRNAs they are carrying to the environment contributing to the further destruction of the disease material. For example, heat generated in the nanosirnas 210 may pass to the cells, thereby contributing to an enhanced effect of the siRNAs released to the environment, causing the cancer cells to die. This combination of mechanical motion, heat production and release of siRNAs to the environment generates a multifaceted attack to the cancer comprising of siRNAs targeting its active cellular pathways, mechanical disruption from the movement of nanosirnas and heat generated from the inductive heating of the nanosirnas.

Furthermore, the poles 204 may be formed from pieces whose gap is adjustable, so as to permit other parts of the body to be treated. It is advantageous to set the gap between the poles 204 to be sufficiently large to permit the part of the body containing the disease material to enter the gap, but not be so large as to reduce the magnetic field strength. Also shown are secondary coils 208 and optional cores 209. Any number of these may be added to modify the distribution of magnetic flux produced by the primary coils and the core. The secondary coils 208 may be wired in series or in parallel with the primary coils, or they can be driven by separate AMF generators. The phase, pulse width and amplitude of the AMF generated by these coils may be adjusted to maximize the field strength in the gap, minimize the field strength in areas which may be sensitive to AMF, or to uniformly distribute the magnetic field strength in a desired manner.

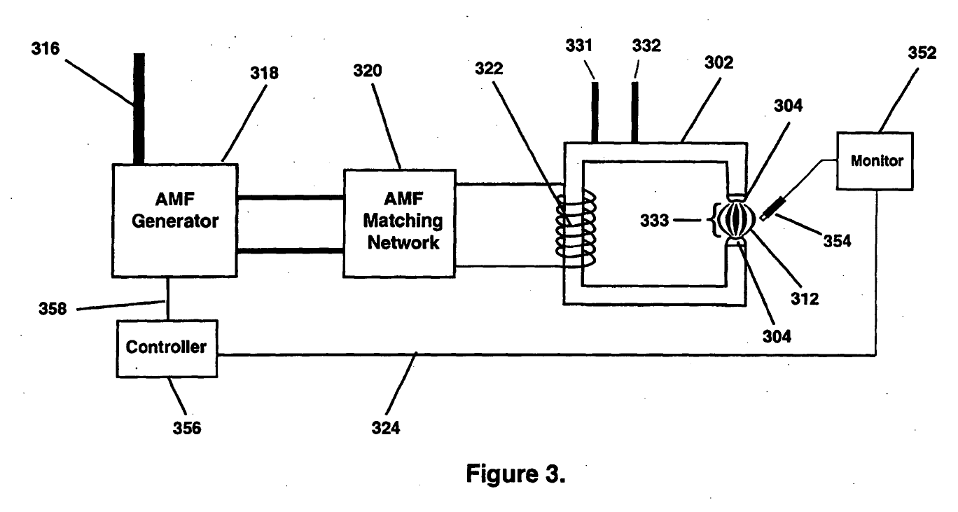

FIG. 3 illustrates a circuit for producing an AMF according to an embodiment of the present invention. The AMF generator 318 is supplied with alternating current (AC) power via the conduit 316. A circulating fluid supply is also provided in the conduit 316. The AMF generator 318 may become hot, and it may be cooled with the circulating fluid supply while in operation. The fluid may be water; however a fluid such as silicone oil or other inorganic or organic fluids with suitable thermal and electric properties may be preferable to increase generator efficiency. The energy produced by the generator 318 is directed through the AMF matching network 320 where the impedance of the generator is matched to the impedance of the coil 322. The impedance of the AMF matching network 320 may be adjustable to minimize the energy reflected back to the generator 318. In another embodiment, the generator frequency may be automatically adjusted to minimize the reflected energy. The modified energy may be directed to the magnetic circuit 302. An AMF is induced in the magnetic circuit 302 as a result of the current passing through the solenoid coil 322. Magnetic lines of flux 312 are produced in the gap 333 between the poles 304 in the magnetic circuit 302. Items 331 and 332 illustrate a liquid cooling send and return.

A feedback loop 324 may be provided for monitoring the magnetic field profile in the gap 333 between the poles 304. The probe 354 may provides data to the monitor 352, which relays information to the controller 356 via an appropriate data bus 324. Information from the controller 356 is relayed to the generator 318 via an appropriate data bus 358. Monitoring the magnetic field profile may be useful in detecting the presence of magnetic particles, monitoring an inductance of tissue, and monitoring the temperature of tissue located in the gap 333.

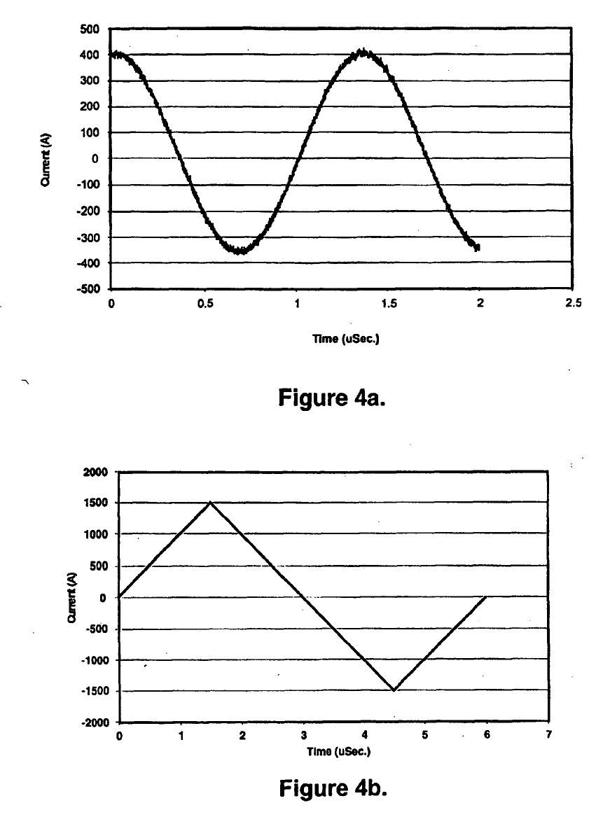

Measuring alternating magnetic fields directly is extremely difficult. Because the AMF is proportional to the current in the coil 322, characteristics of the AMF may be defined in terms of the coil current, which can readily be measured with available test equipment. For example, the coil current may be viewed and measured with a calibrated Rogowski coil and any oscilloscope of suitable bandwidth. The fundamental waveform may be observed as the direct measure of the magnitude and direction of the coil current. Many different types of fundamental waveforms may be used for the AMF. For example, FIG. 4a illustrates a sinusoidal current waveform, and FIG. 4b illustrates a triangular current waveform. The shape of the fundamental waveform may also be square, sawtooth, or trapezoidal.

Most practical generators produce an approximation of these waveforms with some amount of distortion.

For example, FIG. 4a illustrates a sinusoidal current waveform, and FIG. 4b illustrates a triangular current waveform. The shape of the fundamental waveform may also be square, sawtooth, or trapezoidal.

Most practical generators produce an approximation of these waveforms with some amount of distortion. In most applications, this waveform may be nearly symmetrical around zero, as illustrated in FIGS. 4a and 4b. However, there may be a static (DC) current, known as a DC offset, superimposed on the waveform. An AMF with a DC offset can be used to influence the movement of nanosirnas within the body. With a suitable gradient and the “vibration-like” effect of the AC component, the nanosirnas are typically drawn toward the area of highest field strength. FIGS. 4a and 4b show at least one cycle of two different fundamental waveforms with zero or near zero DC offsets. The fundamental period may be defined as the time it takes to complete one cycle. The fundamental frequency may be defined as the reciprocal of the fundamental period. The fundamental frequency may be between 1 kHz and 1 GHz, preferably between 50 kHz and 15 MHz, and more preferably between 100 kHz and 500 kHz. The fundamental frequency may be intentionally modulated, and may often vary slightly as a result of imperfections in the RF generator design.

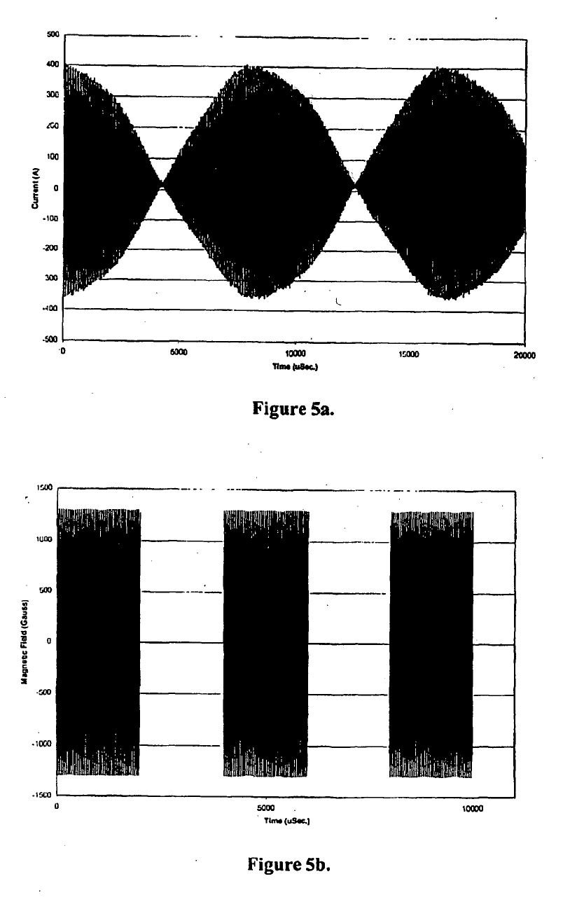

The amplitude of the waveform may also be modulated. FIG. 5a illustrates an embodiment in which a sinusoidal current modulation envelope may be used, and FIG. 5b illustrates an embodiment that utilizes a square modulation envelope. The shape of the amplitude modulation envelope may typically be sinusoidal, square, triangular, trapezoidal or sawtooth, and may be any variation or combination thereof, or may be some other shape.

The AMF produced by the generator may also be pulsed. Pulse width is traditionally defined as the time between the −3 dBc points of the output of a square law crystal detector. Because this measurement technique is cumbersome in this application, we use an alternate definition of pulse width. For the purpose of this invention, pulse width may be defined as the time interval between the 50% amplitude point of the pulse envelope leading edge and the 50% amplitude point of the pulse envelope trailing edge. The pulse width may also be modulated.

The pulse repetition frequency (PRF) is defined as the number of times per second that the amplitude modulation envelope is repeated. The PRF typically lies between 0.0017 Hz and 1000 MHz. The PRF may also be modulated. The duty cycle may be defined as the product of the pulse width and the PRF, and thus is dimensionless. In order to be defined as pulsed, the duty of the generator 318 must be less than unity (or 100%).

The AMF may be constrained to prevent heating healthy tissue to lethal temperatures, for example by setting the temperature of the tissue to be around 43.degree. C., thus allowing for a margin of error of about 3.degree. C. from the temperature of 46.5.degree. C. that is lethal to healthy tissue. This may be accomplished in a variety of ways. The peak amplitude of the AMF may be adjusted. The PRF may be adjusted. The pulse width may be adjusted. The fundamental frequency may be adjusted. The treatment duration may be adjusted.

These four characteristics may be adjusted to maximize the heating rate of the nanosirnas and, simultaneously, to minimize the heating rate of the healthy tissue located within the treatment volume. These conditions may vary depending upon tissue types to be treated, thus the operator may determine efficacious operation levels. In one embodiment, one or more of these characteristics may be adjusted during treatment based upon one or more continuously monitored physical characteristics of tissue in the treatment volume by the probe 354, such as temperature or impedance. This information may then be supplied as input to the generator 318, via the monitor 352, the data bus 324, the controller 356, and the data bus 358 to control output, constituting the feedback loop. In another embodiment, one or more physical characteristics of the nanosirnas (such as magnetic properties) may be monitored during treatment with a suitable device. In this case, one or more magnetic property, such as the magnetic moment, is directly related to the temperature of the magnetic material. Thus, by monitoring some combination of magnetic properties of the nanosirna, the nanosirna temperature can be monitored indirectly. This information may also be supplied as input to the generator 318, via the monitor 352, the data bus 324, the controller 356, and the data bus 358 to control output to become part of the feedback loop. The generator output may be adjusted so that the peak AMF strength is between about 10 and about 10,000 Oersteds (Oe). Preferably, the peak AMF strength is between about 20 and about 3000 Oe, and more preferably, between about 100 and about 2000 Oe.

In another embodiment of the present invention, the differential heating of the nanosirnas, as compared to that of the healthy tissue, may be maximized. The nanosirnas 210 heat in response to each cycle of the AMF. Assuming the fundamental frequency, the PRF, and the pulse width remain constant, the heat output of the nanosirnas 210 continues to increase as peak amplitude of the AMF increases until the magnetic material of the nanosirna reaches saturation. Beyond this point, additional increases in AMF amplitude yield almost no additional heating. At AMF amplitudes below saturation however, it can be said that nanosirna heating is a function of AMF amplitude. Unlike nanosirnas, healthy tissue heating is a result of eddy current flow and a function of the rate of change of the AMF. In particular, the eddy current and resultant tissue heating following the expressions: (1) I.sub.eddy.varies.dB/dT (2) Tissue Heating.varies.I.sub.eddy.sup.2.

From the relationships (1) and (2), it is evident that reducing the rate of change of the AMF yields a significant reduction in tissue heating. In one embodiment of the present invention, this relationship is exploited by using a symmetrical triangular wave, as shown in FIG. 4b, as the fundamental waveform. By avoiding the high rates of change that occur as a sinusoid crosses the X-axis (FIG. 4a), and substituting the constant but lower rate of change associated with a triangular waveform (FIG. 4b), tissue heating may be reduced with little or no sacrifice in nanosirna heating. A triangular waveform, as shown in FIG. 4b, may be achieved by using an appropriate generator, such as a linear amplifier-based generator. Some distortion of the triangle is inevitable, but tangible reductions in tissue heating result from even small reductions in dB/dT.

The heating of both the tissue and the nanosirnas increase with increased AMF amplitude. At low AMF amplitudes, small increases yield significant increases in magnetic heating. As the nanosirnas approach saturation however, their relationship with the AMF amplitude becomes one of diminishing return. This relationship is unique to the particular magnetic material, as are the values that constitute “low” or “saturating” AMF amplitudes. Nanosirna heating is at first related to the AMF amplitude by an exponent >1, which gradually diminishes to an exponent <1 as saturation is approached. At typical pulse widths and duty cycles, eddy current heating is directly related to duty cycle. The capability to pulse the generator output, as illustrated in FIG. 5a or 5b, allows the benefits of operating at higher AMF amplitudes while maintaining a constant reduced tissue heating by reducing the duty cycle.

It is desirable to apply the AMF to the treatment area 205 of the patient 105. Generating high peak amplitude AMF over a large area requires a very large AMF generator and exposes large amounts of healthy tissue to unnecessary eddy current heating. Without some way of directing the field to where it is useful, disease in the chest or trunk may only be practically treated by placing the patient within a large solenoid coil. This would expose most of the major organs to eddy current heating, which must then be monitored and the AMF adjusted so as not to overheat any part of a variety of tissue types. Each of these tissue types has a different rate of eddy current heating. The peak AMF strength would need to be reduced to protect those tissue types that experience the most extreme eddy current heating. If the varieties of exposed tissue are minimized, it is likely that the AMF strength can be increased, and thereby reducing the treatment time and increasing the efficacy. One method of confining the high peak amplitude AMF to treatment area 205 is by defining the lowest reluctance path of magnetic flux with high permeability magnetic material. This path is referred to as a magnetic circuit (102 in FIGS. 1 and 302 in FIG. 3). The magnetic circuit may be provided so that all or most of the magnetic flux produced by the coil 322 may be directed to the treatment area 205. One benefit of the magnetic circuit 302 is that the necessary amount of flux may be reduced since the amount of flux extending beyond the treatment area 205 is minimized. Reducing the required flux reduces the required size and power of the AMF generator, and minimizes exposure of tissue outside the treatment area 205 to high peak amplitude AMF. In addition, a reduced area of AMF exposure avoids the unintentional heating of surgical or dental implants and reduces the likelihood that they will need to be removed prior to treatment, thereby avoiding invasive medical procedures. Concentrating the field permits the treatment of large volumes within the chest or trunk with a portable size device.

The material used to fabricate the magnetic circuit 302 may be appropriate to the peak amplitude and frequency of the AMF. The material may be, but is not limited to, iron, powdered iron, assorted magnetic alloys in solid or laminated configurations and ferrites. The pole faces 104, 204, and 304 may be shaped and sized to further concentrate the flux produced in the treatment area. The pole faces 304 may be detachable. Different pole pieces having different sizes and shapes may be used, so that the treatment area and volume may be adjusted. When passing from one material to another, the lines of magnetic flux 312 travel in a direction normal to the plane of the interface plane. Thus, the face 304 may be shaped to influence the flux path through gap 333. The pole faces 304 may be detachable and may be chosen to extend the magnetic circuit 302 as much as possible, to minimize gap the 333 while leaving sufficient space to receive that portion of the patient being treated. As discussed above, the addition of secondary coils can aid in the concentration of the field as well as reducing the field strength in sensitive areas.

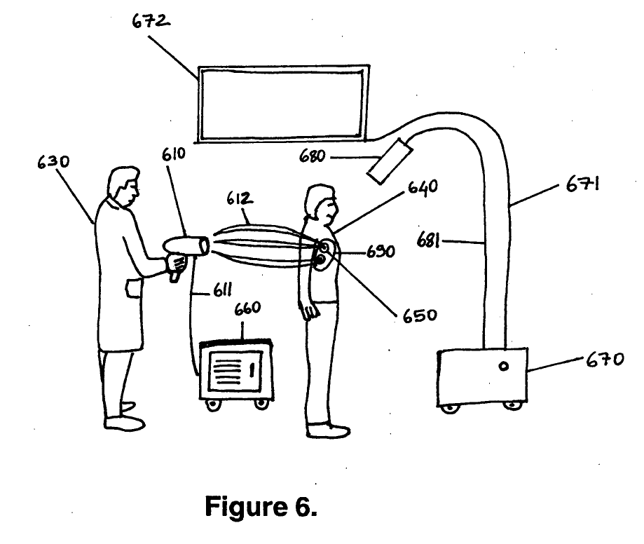

FIG. 6 schematically illustrates a handheld medical device and therapy system configuration according to an embodiment of the present invention. Operator 630 holds a hanheld electromagnetic wave gun 610 which he or she can direct towards the target area of interest of a patient 640. Electromagnetic wave gun 610 has a electromagnetic wave generator unit 660 and connected to it with a cord 611.

Electromagnetic waves 612 are directed towards target area 690 where nanosirna 650 particles have accumulated with the magnetic field created by the magnetic field generator and computer unit 670 and probe 680, connector cord 681. Probe 680 has capability of monitoring the location and concentration of nanosirnas 650. This can be visualized by the operator 630 on the computer screen 672. Computer screen 672 is connected to the magnetic field generator and computer unit 670 by a cord 671.

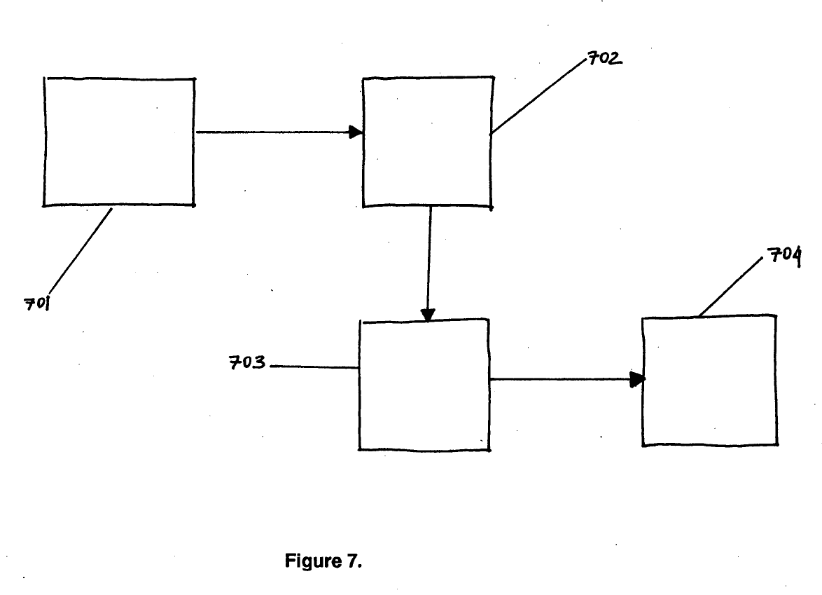

FIG. 7. schematically shows the procedure to individualize a patient's treatment based on the gene expression profile of the predetermined target. Nanosirna particles will be carrying siRNAs which are chosen based upon the gene expression profile of the predetermined target. In this illustrative example, predetermined target is lung cancer. Lung cancer is diagnosed with biopsy of the tumor detected by a medical imaging modality. Medical imaging modality can be computerized tomography (CT), magnetic resonance imaging (MRI), positron emission tomography (PET) or simply a chest radiograph (Chest XRay). Gene expression profiling of the lung cancer is performed as step one 701. In addition to gene expression profiling, proteomic analysis of the lung cancer can also be performed to help determining the most active cellular pathways of the target. Gene expression profiling of the cancer will generate a report of most active genes in this particular cancer. Based upon this report of gene expression profile, siRNA constructs can be formed or selected from a preformed library to use for treating this particular cancer as the second step 702. Gene expression profile of this tumor and proteomic analysis of it, give significant clues about most active cellular pathways contributing to the cancer's growth and resistance to the body's immune defense mechanisms. Cancer develops resistance mechanisms and evades the immune system. It is usually not successful to attack a single cellular pathway to destroy the cancer cell, because of rapid development of resistance by the cancer to that single agent. Therefore, it is desirable to attack the cancer at multiple active cellular pathways. Active cellular pathways keeping the cancer alive and thriving may also differ from cancer to cancer, from individual to individual. Cancer by itself is usually not homogenous either but consists of various cell types at different levels of degeneration. Dysplasia is a term to define the level of degeneration at early phases of cancer. It is therefore desirable to define a cancer's gene expression profile signature and design a treatment specifically effective for this particular cancer. After construction or selection of the desired nanosirna particles, patient receives these nanoparticles which may be but not limited to intravenously, orally, intramuscularly or subcutaneously as step 3. During and after step 3 of the procedure 703, patient may be exposed to a magnetic field to concentrate and increase the number of nanosirna particles at the predetermined target tissue. Last step is to apply energy to the target area which can be in the form of electromagnetic waves to excite nanosirna particles 704. This leads to release of siRNA and its fragments into the target tissue milieu. Mechanical disruption from the motion of nanosirna particles, heat generation from the inductive heating of nanosirna particles and release of siRNA targeted to the target in question create a multifaceted attack to the target. In one embodiment of this invention, target is cancer and this procedure leads to destruction of cancer.

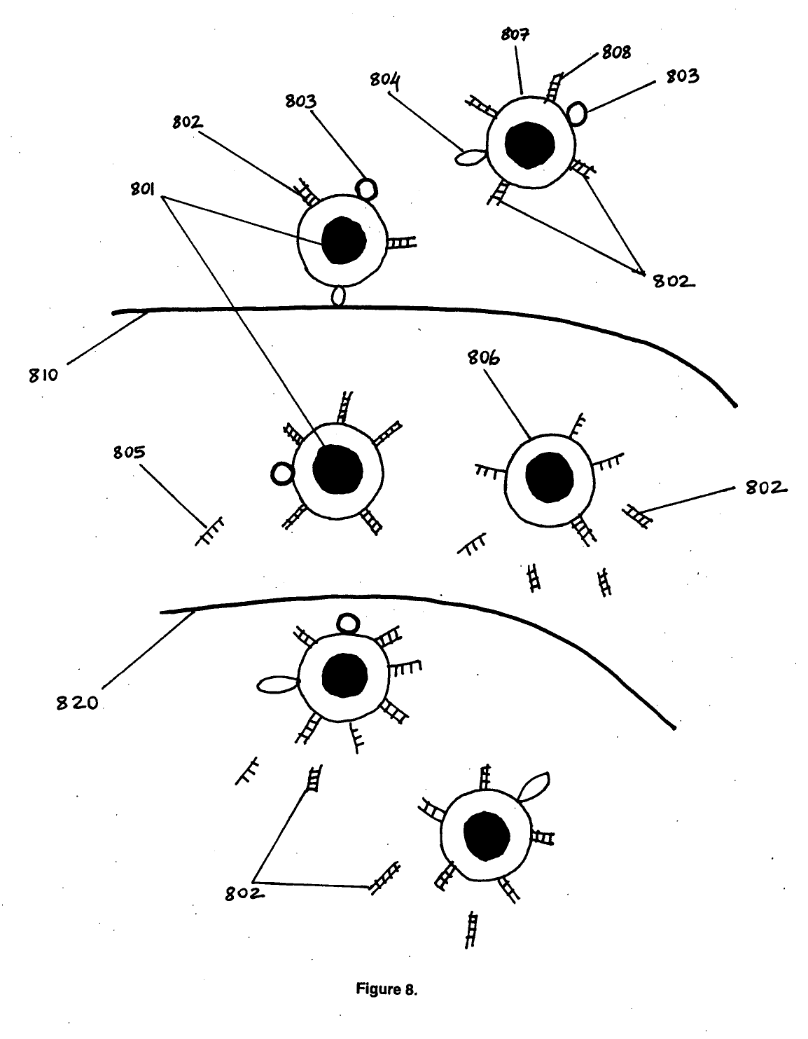

FIG. 8 discloses a nanosirna configuration according to an embodiment of the present invention. A spherical shaped nanosirna 807, having a magnetic energy susceptor core particle 801 at its center, is shown. The magnetic energy suceptor core particle 801 may be covered with a coating 806. At least one short interfering RNA (siRNA) 802 or micro interfering RNA (mRNA) 808, selected based upon the gene expression profile of the predetermined target, may be located on either interior or exterior portion of the nanosirna 807. The short interfering RNA (siRNA) 802 or micro interfering RNA (mRNA) 808 may be selected to downregulate the gene expression of a particular gene of the predetermined target which can be a type of cell or disease matter. Heat is generated when the magnetic energy susceptor core particle 801 of the nanosirna 807 is subjected to the AMF. In a general sense, this heat represents an energy loss as the magnetic properties of the material are forced to oscillate in response to the applied alternating magnetic field. The amount of heat generated per cycle of magnetic field and the mechanism responsible for the energy loss depend on the specific characteristics of both the magnetic material of the energy susceptor core particle 801 and the magnetic field. The magnetic energy susceptor core particle 801 heats to a unique temperature, known as the Curie temperature, when subjected to the AMF. The Curie temperature is the temperature of the reversible ferromagnetic to paramagnetic transition of the magnetic material. Below this temperature, the magnetic material heats in an applied AMF. However, above the Curie temperature, the magnetic material becomes paramagnetic and its magnetic domains become unresponsive to the AMF. Thus, the material does not generate heat when exposed to the AMF above the Curie temperature. As the material cools to a temperature below the Curie temperature, it recovers its magnetic properties and resumes heating, as long as the AMF remains present. This cycle may be repeated continuously during exposure to the AMF. Thus, magnetic materials are able to self-regulate the temperature of heating. The temperature to which the magnetic energy susceptor core particle 801 heats is dependent upon, inter alia, the magnetic properties of the material, characteristics of the magnetic field, and the cooling capacity of the target site 214. Selection of the magnetic material and AMF characteristics may be tailored to optimize treatment efficacy of a particular tissue or target type. In an embodiment of the present invention, the magnetic material may be selected to possess a Curie temperature between 40.degree. C. and 150.degree. C.

The magnetic attributes of ferromagnets, ferrites (ferrimagnets), and superparamagnets are determined by an ensemble of interacting magnetic moments in a crystalline structure. The magnetic moments of ferromagnets are parallel and equal in magnitude, giving the material a net magnetization, or net magnetization vector. By contrast, ferrites are ferrimagnetic, where adjacent magnetic moments are parallel in direction and unequal in magnitude, yielding a net magnetization in ferrimagnetic coupling. Superparamagnets possess clusters or collections of atomic magnetic moments that are either ferromagnetic or ferrimagnetic, however there may be no particular relationship in the orientation of the moments among several clusters. Thus, a superparamagnetic material may possess a net magnetic moment.

A magnetic domain may be defined as an area of locally saturated magnetization, and the magnetic domain boundary thickness, or the distance separating adjacent magnetic domains, may be about 100 nm. Thus, magnetic particles (ferromagnetic or ferrimagnetic) possessing a dimension smaller than 250 nm, and preferably less than about 100 nm, are single domain magnetic particles, where each particle is a magnetic dipole.

The mechanisms responsible for energy loss exhibited by single domain particles exposed to an alternating magnetic field are still not well understood, however a currently accepted description exists, which is included herein for clarity. When a single domain particle is exposed to an AMF, the whole magnetic dipole rotates in response to the field with a concomitant energy loss liberated as heat. This mechanism is often referred to as the Neel mechanism. The external magnetic forces required for this intrinsic change in magnetization depend upon the anisotropy energy of the magnetic domain, size, and shape of the single domain particle. Furthermore, it is currently accepted that there is a mechanical rotation of the entire single domain particle when exposed to an alternating magnetic field. This latter phenomenon, commonly called the Brownian mechanism, also contributes to the energy loss of a single domain particle, and is proportional to the viscosity of the material surrounding the particle. Thus, the coating 806 may enhance the heating properties of the nanosirna 807, particularly if the coating has a high viscosity, for example, if the coating is a polymer.

The heating mechanism responsible for the energy loss experienced by a single domain particle in an AMF can be clearly distinguished from the hysteresis heating of larger, or multidomain magnetic particles. Single domain particles of a given composition can produce substantially more heat per unit mass than multi-domain particles that are 1000 times larger (multi domain particles). The heating mechanism exhibited by single domain particles may be optimized to produce superior heating properties over larger particles for disease treatment. The amount of heat delivered to a cell may be tailored by controlling both the particle size and coating variation, as well as characteristics of the magnetic field, thereby providing a range of possible nanosirna compositions designed for material-specific treatments.

Many aspects of the magnetic energy susceptor core particle 801, such as material composition, size, and shape, directly affect heating properties. Many of these characteristics may be designed simultaneously to tailor the heating properties for a particular set of conditions found within a tissue type. For example, first considering the magnetic energy susceptor core particle 801, the most desirable size range depends upon the particular application and on the material(s) comprising the magnetic energy susceptor core particle 801.

The size of the magnetic energy susceptor core particle 801 determines the total size of the nanosirna 807. Nanosirnas 807 that are to be injected may be spherical and may be required to have a long residence time in the bloodstream, i.e., avoid sequestration by the liver and other non-targeted organs. The nanosirna 807 may be successful in avoiding sequestration if its total diameter is less than about 30 nm. If the nanosirna 807 contains a magnetite (Fe.sub.3O.sub.4) energy susceptor core particle 801, then a preferred diameter of the magnetic energy susceptor core particle 801 may be between about 8 nm and about 20 nm. In this case, the nanosirnas 807 may be sufficiently small to evade the liver, and yet the magnetic energy susceptor core particle 801 still retains a sufficient magnetic moment for heating. Magnetite particles larger than about 8 nm generally tend to be ferrimagnetic and thus appropriate for disease treatment. If other elements, such as cobalt, are added to the magnetite, this size range can be smaller. This results directly from the fact that cobalt generally possesses a larger magnetic moment than magnetite, which contributes to the overall magnetic moment of the cobalt-containing magnetic energy susceptor core particle 801. In general, the preferred size of the nanosirna 807 may be about 0.1 nm to about 250 nm, depending upon the disease indication and nanosirna 807 composition.

While determining the size of the magnetic energy susceptor core particle 801, its material composition may be determined based on the particular predetermined target. Because the self-limiting temperature of a magnetic material, or the Curie temperature, is directly related to the material composition, as is the total heat delivered, magnetic particle compositions may be tuned to different tissue or target types. This may be required because each target type, given its composition and location within the body, possesses unique heating and cooling capacities. For example, a tumor located within a region that is poorly supplied by blood and located within a relatively insulating region may require a lower Curie temperature material than a tumor that is located near a major blood vessel. Targets that are in the bloodstream will require different Curie temperature materials as well. Thus, in addition to magnetite, particle compositions may contain elements such as cobalt, iron, rare earth metals, etc.

The presence of the coating 806 and the composition of the coating material may form an integral part of the energy loss, and thus the heat produced, by the nanosirna 807. In addition, the coating 806 surrounding the particles may serve additional purposes. Its most important role may be to provide a biocompatible layer separating the magnetic material from the immunologic defenses in a patient, thereby controlling the residence time of the particles in the blood or tissue fluids. Another important role the coating may be the release of siRNAs at the predetermined target area as a result of heating and motion of the nanosirna 807 by energy transfer through electromagnetic waves. If the magnetic energy suceptor core particle 801 is a nanoshell, energy transfer to the nanosirna 807 may be done by using infrared light. This infrared light can be used in the form of a laser beam to direct towards and target the predetermined target area. Coating 806 can also dissolve and disintegrate in the presence of heat leading to the release of siRNA constructs 802 attached to it.

In addition, the coating 806 may serve to protect the siRNA constructs 802 from degradation inside the body by the enzymes. siRNA constructs 802 are degraded quickly by enzymes inside the body. siRNA constructs 802 may be chemically modified to prevent degradation inside the body. Coating 806 may also serve as protective coating siRNA constructs 802 from enzymatic or any other type of degradation inside the human body. Thus, siRNA constructs 802 may be able to reach the predetermined target without being destroyed by the enzymes in the body. A second function of the coating 806 materials may be the prevention of particle aggregation, as the nanosirnas 807 may be suspended in a fluid. It may be also be advantageous to coat the nanosirnas 807 with a biocompatible coating that is biodegradable. In such an application, both the coating 806 and the magnetic energy susceptor core particle 801 may be digested and absorbed by the body.

Suitable materials for the coating 806 include synthetic and biological polymers, copolymers and polymer blends, and inorganic materials. Polymer materials may include various combinations of polymers of acrylates, siloxanes, styrenes, acetates, akylene glycols, alkylenes, alkylene oxides, parylenes, lactic acid, polyethylene glycol and glycolic acid. Further suitable coating materials include a hydrogel polymer, a histidine-containing polymer, and a combination of a hydrogel polymer and a histidine-containing polymer.

Non-biodegradable or biodegradable polymers may be used to form the nanosirna 807 particles. In the preferred embodiment, the coating 806 of nanosirna 807 particles are formed of a biodegradable polymer. Non-biodegradable polymers may be used for oral administration. In general, synthetic polymers are preferred, although natural polymers may be used and have equivalent or even better properties, especially some of the natural biopolymers which degrade by hydrolysis, such as some of the polyhydroxyalkanoates. Representative synthetic polymers are: poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivativized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulfate sodium salt jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) jointly referred to herein as “polyacrylic acids”), poly(butyric acid), poly(valeric acid), and poly(lactide-co-caprolactone)-, copolymers and blends thereof. As used herein, “derivatives” include polymers having substitutions, additions of chemical groups and other modifications routinely made by those skilled in the art.

Examples of preferred biodegradable polymers include polymers of hydroxy acids such as lactic acid and glycolic acid, and copolymers with PEG, polyanhydrides, poly(ortho)esters, polyurethanes, poly(butyric acid), poly(valeric acid), poly(lactide-co-caprolactone), blends and copolymers thereof.

Examples of preferred natural polymers include proteins such as albumin, collagen, gelatin and prolamines, for example, zein, and polysaccharides such as alginate, cellulose derivatives and polyhydroxyalkanoates, for example, polyhydroxybutyrate. The in vivo stability of the nanosirna 807 particles can be adjusted during the production by using polymers such as poly(lactide-co-glycolide) copolymerized with polyethylene glycol (PEG). If PEG is exposed on the external surface, it may increase the time these materials circulate due to the hydrophilicity of PEG.

Examples of preferred non-biodegradable polymers include ethylene vinyl acetate, poly(meth)acrylic acid, polyamides, copolymers and mixtures thereof.

In a preferred embodiment, PEG is used as the biodegradable polymer for the coating 806 of nanosirna 807. At the time of this invention, best use method is thought to be nanosirna 807 with a magnetite magnetic energy susceptor core particle 801 and PEG used as the biodegradable polymer for the coating 806 of the nanosirna 807. Energy source is an alternating magnetic field 320 as best method of use at the time of this invention.

Coating materials may include combinations of biological materials such as a polysaccharide, a polyaminoacid, a protein, a lipid, a glycerol, and a fatty acid. Other biological materials for use as a coating material may be a heparin, heparin sulfate, chondroitin sulfate, chitin, chitosan, cellulose, dextran, alginate, starch, carbohydrate, and glycosaminoglycan. Proteins may include an extracellular matrix protein, proteoglycan, glycoprotein, albumin, peptide, and gelatin. These materials may also be used in combination with any suitable synthetic polymer material.

Inorganic coating materials may include any combination of a metal, a metal alloy, and a ceramic. Examples of ceramic materials may include a hydroxyapatite, silicon carbide, carboxylate, sulfonate, phosphate, ferrite, phosphonate, and oxides of Group IV elements of the Periodic Table of Elements. These materials may form a composite coating that also contains any biological or synthetic polymer. Where magnetic energy susceptor core particle 801 is formed from a magnetic material that is biocompatible, the surface of the particle itself operates as the biocompatible coating.

The coating material may also serve to facilitate transport of the nanosirna 807 into a cell, a process known as transfection. TAT peptide 804 may be used in the coating 806 to facilitate intracellular transfer of the nanosirna 807. Such coating materials, known as transfection agents, include vectors, prions, polyaminoacids, cationic liposomes, amphiphiles, and non-liposomal lipids or any combination thereof. A suitable vector may be a plasmid, a virus, a phage, a viron, a viral coat. The nanosirna coating may be a composite of any combination of transfection agent with organic and inorganic materials, such that the particular combination may be tailored for a particular type of a disease material and a specific location within a patient's body. Nanosirna 807 may enter into the cell through the cell membrane 810 and into the nucleus of the cell through the nuclear membrane 820 of the cell. Fragments such as single stranded siRNA 805 may be released into the cell.

To ensure that the nanosirna 807 selectively accumulates at the predetermined target area, an appropriate ligand 803 may be combined with the nanosirna 807. The association of a ligand or ligands with the nanosirnas 807 allows for targeting of cancer or disease markers on cells. It also allows for targeting biological matter in the patient The term ligand relates to compounds which may target molecules including, for example, proteins, peptides, antibodies, antibody fragments, saccharides, carbohydrates, glycans, cytokines, chemokines, nucleotides, lectins, lipids, receptors, steroids, neurotransmitters, Cluster Designation/Differentiation (CD) markers, and imprinted polymers and the like. The preferred protein ligands include, for example, cell surface proteins, membrane proteins, proteoglycans, glycoproteins, peptides and the like. The preferred nucleotide ligands include, for example, complete nucleotides, complimentary nucleotides, and nucleotide fragments. The preferred lipid ligands include, for example phospholipids, glycolipids, and the like. The ligand 803 may be covalently bonded to or physically interacted with the magnetic energy susceptor core particle 801 or the coating 806. The ligand 803 may be bound covalently or by physical interaction directly to an uncoated portion of the magnetic energy susceptor core particle 801. The ligand 803 may be bound covalently or by physical interaction directly to an uncoated portion of the magnetic energy susceptor core particle 801 and partially covered by the coating 806. The ligand 803 may be bound covalently or by physical interaction to a coated portion of the nanosirna 807. The ligand 803 may be intercalated to the coated portion of the nanosirna 807.

Covalent bonding may be achieved with a linker molecule. The term “linker molecule,” as used herein, refers to an agent that targets particular functional groups on the ligand 803 and on the magnetic energy susceptor core particle 801 or the coating 806, and thus forms a covalent link between any two of these. Examples of functional groups used in linking reactions include amines, sulfhydryls, carbohydrates, carboxyls, hydroxyls and the like. The linking agent may be a homobifunctional or heterobifunctional crosslinking reagent, for example, carbodiimides, sulfo-NHS esters linkers and the like. The linking agent may also be an aldehyde crosslinking reagent such as glutaraldehyde. The linking agent may be chosen to link the ligand 803 to the magnetic energy susceptor core particle 801 or the coating 806 in a preferable orientation, specifically with the active region of the ligand 803 available for targeting the predetermined target. Physical interaction does not require the linking molecule and the ligand 803 be bound directly to the magnetic energy susceptor core particle 801 or to the coating 806 by non-covalent means such as, for example, absorption, adsorption, or intercalation.

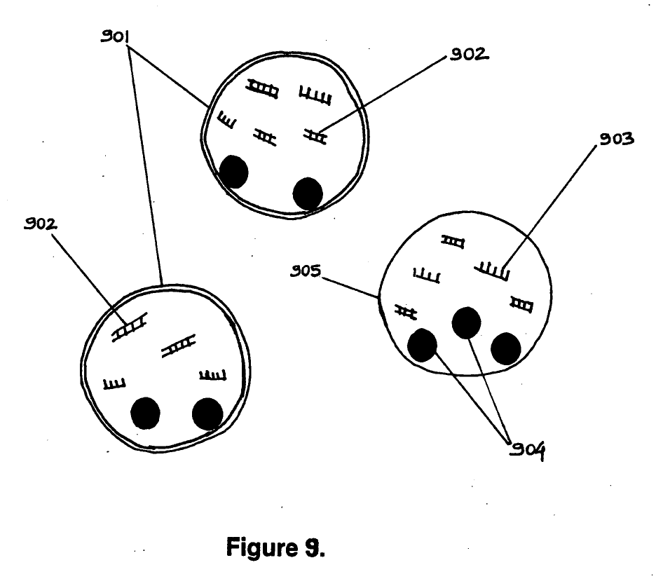

FIG. 9 schematically illustrates a composition of nanosirna according to an embodiment of the present invention. Nanosirna 905 particles may be in a solid or semisolid state with siRNA 904 and energy susceptible magnetic core particles 904 embedded into them. Energy transfer from an energy source dissolves or melts the coating 901 material encasing the siRNA 902 particles, single stranded siRNA fragments 903 and energy susceptible magnetic core particles 904. This leads to their release to the target environment. Nanosirna 905 particles may also be hollow spheres carrying the siRNA 902 particles, single stranded siRNA fragments 903 and energy susceptible magnetic core particles 904.

I claim:

1) A medical device comprising:
a) at least one energy susceptible nanoscale particle with magnetic properties administered to at least a portion of a subject comprising a predetermined target; and
b) said nanoscale particle has at least one nucleotide sequence; and
c) said nucleotide sequence is selected based on gene expression profile of the predetermined target.

2) A medical device according to claim 1, wherein the nucleotide sequence is a short interfering ribonucleic acid (RNA) (siRNA).

3) A medical device according to claim 1, wherein the nucleotide sequence is a micro interfering ribonucleic acid (RNA) (mRNA).

4) A medical device according to claim 1, wherein the nucleotide sequence is a single strand of a short interfering ribonucleic acid (RNA) (siRNA).

5) A medical device according to claim 1, wherein the predetermined target is associated with a cancer.

6) A medical device according to claim 1, wherein the predetermined target is a an infectious agent such as a bacteria, virus, fungus, parasite.

7) A medical device according to claim 1, wherein the predetermined target is associated with an infection.

8) A medical device according to claim 1, wherein the predetermined target is associated with an autoimmune disease.

9) A medical device according to claim 1, wherein the predetermined target is cancer.

10) A medical device according to claim 1, wherein the nanoscale particle has a biocompatible coating.

11) A medical device according to claim 1, wherein the nanoscale particle has an energy susceptor core.

12) A medical device according to claim 11, wherein the nanoscale particle with energy susceptor core has magnetic properties.

13) A medical device according to claim 11, wherein the energy susceptor core is a nanoshell.

14) A medical device comprising:
a) at least one energy susceptible nanoscale particle with magnetic properties administered to at least a portion of a subject comprising a predetermined target; and
b) said nanoscale particle has at least one nucleotide sequence; and
c) said nucleotide sequence is selected based on gene expression profile of the predetermined target, wherein said medical device has means to apply a magnetic field to the target during and after delivery of the nanoscale particle to localize the nanoscale particle and increase their concentration at the target.

15) A medical device according to claim 14, wherein the nanoscale particle magnetic property is magnetism.

16) A medical device according to claim 14, wherein the nanoscale particle magnetic property is paramagnetism.

17) A medical device according to claim 14, wherein the nanoscale particle has biocompatible coating.

18) A medical device according to claim 14, wherein energy from an energy source in the form of electromagnetic wave, alternating magnetic field, microwave, acoustic, infrared light, or any combination of thereof, is transferred to the nanoscale particle to provide heating and motion of said nanoscale particle.

19) A medical device according to claim 18, wherein albumin or albumin derivative proteins are attached to the nanoscale particle.

20) A medical device according to claim 18, wherein TAT peptide is attached to the nanoscale particle.

21) A medical device according to claim 1, wherein the nucleotide sequence is a chemically modified short interfering ribonucleic acid (RNA) (siRNA).

22) A medical device according to claim 14, wherein the nucleotide sequence is a chemically modified short interfering ribonucleic acid (RNA) (siRNA).
